(19) United States
(12) Patent Application Publication
(10) Pub. No.: US 2023/0159476 A1
(43) Pub. Date: May 25, 2023

(54) PROCESS FOR THE SYNTHESIS OF COMPOUNDS WHICH ABSORB ULTRAVIOLET RADIATION IN FLOW CONDITIONS AND FORMULATIONS COMPRISING SAME

(71) Applicant: ELIKIMIA INC., Montreal (CA)

(72) Inventors: Elie Abou KHALIL, Rosemere (CA); Renan CARIOU, Montreal (CA); Shawn PARISIEN-COLLETTE, Montreal (CA); Julie CHANTEAU, Montreal (CA); Sophie LANGIS-BARSETTI, Montreal (CA)

(21) Appl. No.: 17/919,394

(22) PCT Filed: Feb. 26, 2021
(86) PCT No.: PCT/CA2021/050248

§ 371 Date: Oct. 17, 2022

Related U.S. Application Data
(60) Provisional application No. 62/981,755, filed on Feb. 26, 2020.

(51) Int. Cl. C07D 279/14; C07C 211/49; A61K 8/49; A61K 8/41; A61K 8/04; A61Q 17/04

(52) U.S. Cl. CPC C07D 279/14; C07C 211/49; A61K 8/4933; A61K 8/411; A61K 8/042; A61Q 17/04

(57) ABSTRACT

The present invention generally relates to a chemical process under flow conditions for manufacturing compounds which absorb ultraviolet (UV) radiation and protect biological materials as well as non-biological materials from damaging exposure to UV radiation. The present invention further includes formulations and compositions comprising such compounds for use in absorbing UV radiation, as well as methods for protecting biological materials as well as non-biological materials from damaging exposure to UV radiation.

### CROSS-REFERENCE TO RELATED APPLICATIONS

The present patent application claims the benefits of priority of U.S. Patent Application No. 62/981,755, entitled “PROCESS FOR THE SYNTHESIS OF COMPOUNDS WHICH ABSORB ULTRAVIOLET RADIATION IN FLOW CONDITIONS AND FORMULATIONS COMPRISING SAME” and filed at the United States Patent Office on Feb. 26, 2020, the content of which is incorporated herein by reference.

### FIELD OF THE INVENTION

The present invention generally relates to a flow chemical process for making compounds which absorb ultraviolet (UV) radiation and protect biological materials as well as non-biological materials from damaging exposure to UV radiation and formulations comprising same.

### BACKGROUND OF THE INVENTION

Commercially available ultraviolet blocking agents typically include compounds such as para-aminobenzoic acid derivatives, benzotriazoles, benzophenones, methoxycinnamates and salicylates. Mycosporine-like amino acids (MAAs) have also been identified as ultraviolet-absorbing agents. MAAs are small molecules of about 400 Da produced by organisms that live in environments with high volumes of sunlight, typically marine environments. The structures of over 30 MAAs have been resolved and they contain a central cyclohexenone or cyclohexenimine ring as well as a wide variety of substitutions. The ring structure is thought to absorb ultraviolet light and accommodate free radicals. MAAs absorb ultraviolet light, typically between 310 nm and 360 nm. It is this light absorbing property that allows MAAs to protect cells from harmful ultraviolet radiation. Biosynthetic pathways of specific MAAs depend on the specific MAA and the organism that is producing it. These biosynthetic pathways often share common enzymes and intermediates with other major biosynthetic pathways.

Useful ultraviolet absorbing agents such as the ones mentioned above must meet various criteria including stability, acceptable permanence, efficacy, compatibility with the media with which they are to be mixed or be incorporated into, non-toxicity and not harmful to the surface onto which they are to be applied. These criteria limit the choice of ultraviolet protecting agents available to be used in various applications. Some such agents are described in U.S. Pat. No. 9,487,474 owned by the applicant herein.

Therefore, there remains a need in the art for additional agents that meet these criteria, that absorb ultraviolet radiations and that protect biological and non-biological materials against the harmful damages caused by ultraviolet radiations and for a process to produce such agents under flow conditions.

### SUMMARY OF THE INVENTION

The shortcomings of the prior art are generally mitigated by a new chemical process to manufacture compounds under flow conditions. These compounds absorb UV radiation and protect biological materials as well as non-biological materials from damaging exposure to UV radiation. The process for the synthesis of these compounds is performed in flow conditions.

Other and further aspects and advantages of the present invention will be obvious upon an understanding of the illustrative embodiments about to be described or will be indicated in the appended claims, and various advantages not referred to herein will occur to one skilled in the art upon employment of the invention in practice.

### BRIEF DESCRIPTION OF THE DRAWINGS

The above and other objects, features and advantages of the invention will become more readily apparent from the following description, reference being made to the accompanying drawings in which:

FIG. 1 is a graph plotting the evolution of level absorption as a function of the wavelength for various actives.

FIG. 2 is a graph illustrating the evolution of SPF as a function of the percentage of actives.

### DETAILED DESCRIPTION OF THE PREFERRED EMBODIMENT

### Definitions

The terms “comprising” and “including”, as used herein, unless otherwise indicated, are used in their open, non limiting sense.

As used herein, the terms “compound” and “compound(s) of the invention” are used interchangeably to refer to any compounds, including acceptable salts, hydrates or solvates thereof, disclosed herein specifically or generically.

The expression “biological materials”, as used herein, unless otherwise indicated, is intended to include humans, animals and plants and includes for example: cells, hair, skin, as well as other human and animal tissues. The expression “non-biological materials”, as used herein, unless otherwise indicated, is intended to include all things that do not fall into the definition of “biological materials”.

The expression “solar radiation”, as used herein, unless otherwise indicated, is intended to include the total frequency spectrum of electromagnetic radiation given off by the sun, including radio waves, X-rays, infrared, visible, and ultraviolet.

The terms “ultraviolet” and “UV”’, as used herein, unless otherwise indicated, are intended to mean ultraviolet or ultraviolet light. UV is electromagnetic radiation with a wavelength shorter than that of visible light, but longer than X-rays, in the range of about 10 nm to about 400 nm, and energies from about 3 eV to about 124 eV (the abbreviation “eV, herein refers to electron volts). Ultraviolet A (UVA) refers to UV radiation in the spectrum of between 320-400 nm, it is also referred to as “longer” rays. The UVA waveband is further divided into UVAI (340-400 nm) and UVA II (320-340 nm). UVA are the principal cause of long-term skin damage due to the sun and may also contribute to sunburn. Ultraviolet B (UVB) refers to radiation in the spectrum of 290-320 nm, it is also referred to as “shorter” rays. UVB rays are the principal cause of sunburn due to sun exposure.

The term “imine” or “imino”, as used herein, unless otherwise indicated, includes a functional group or chemical compound containing a carbon-nitrogen double bond. The expression “imino compound”, as used herein, unless otherwise indicated, refers to a compound that includes an “imine” or an “imino” group as defined herein.

The term “hydroxyl”, as used herein, unless otherwise indicated, includes —OH. The terms “halogen”’ and “halo”, as used herein, unless otherwise indicated, include a chlorine, chloro, Cl; fluorine, fluoro, F, bromine, bromo, Br; or iodine, iodo, I.

The term “aryl”, as used herein, unless otherwise indicated, includes a carbocyclic aromatic group. Examples of aryl groups include, but are not limited to, phenyl, benzyl, naphthyl and anthracenyl.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group.

The term “alkyl” as used herein, unless otherwise indicated, includes saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl. -n-butyl, -n-pentyl, -n-hexyl, -n-heptyl and -n-octyl, while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C—Cs alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, -3-methyl-1 butynyl.

The term “carboxyl”, as used herein, unless otherwise indicated, includes a functional group consisting of a carbon atom double bonded to an oxygen atom and single bonded to a hydroxyl group (—COOH).

The term “alkenyl”, as used herein, unless otherwise indicated, includes alkyl moieties having at least one carbon-carbon double bond wherein alkyl is as defined above and including E and Z isomers of said alkenyl moiety.

The term “alkynyl”, as used herein, unless otherwise indicated, includes alkyl moieties having at least one carbon-carbon triple bond wherein alkyl is as defined above. The term “acyl”, as used herein, unless otherwise indicated, includes a functional group derived from an aliphatic carboxylic acid, by removal of the hydroxyl (—OH) group.

The term “alkoxyl’, as used herein, unless otherwise indicated, includes O-alkyl groups wherein alkyl is as defined above and O represents oxygen. Representative alkoxyl groups include, but are not limited to. —O-methyl, —O-ethyl, —O-n-propyl. —O-n-butyl, —O-n-pentyl, —O-n-hexyl, —O-n-heptyl, —O-n-octyl, —O-isopropyl. —O-sec-butyl, —O— isobutyl, —O-tert-butyl, —O-isopentyl, —O-2-methylbutyl, —O-2-methylpentyl, —O-3-methyl pentyl, —O-2,2-dimethylbutyl, —O-2,3-dimethylbutyl, —O-2,2-dimethylpentyl, —O-2,3-dimethylpentyl, —O-3.3-dimethylpentyl, —O-2,3,4-trimethylpentyl, —O-3-methyl hexyl, —O-2,2-dimethylhexyl, —O-2,4-dimethylhexyl, —O-2,5-dimethylhexyl, —O-3,5-dimethylhexyl, —O-2, 4.dimethylpentyl, O-2-methylheptyl, —O-3-methylheptyl, —O-vinyl, —O-allyl, —O-1-butenyl, O-2-butenyl, —O-isobutylenyl, —O-1-pentenyl, —O-2-pentenyl, —O-3-methyl-1-butenyl, —O-2-methyl-2-butenyl, —O-2,3-dim ethyl-2-butenyl, —O-1-hexyl, —O-2-hexyl, —O-3-hexyl, —O-acetylenyl, —O— propynyl, —O-1-butynyl. —O-2-butynyl. —O-1-pentynyl. —O-2-pentynyl and —O-3-methyl 1-butynyl, —O-cyclopropyl. —O-cyclobutyl, —O-cyclopentyl, —O-cyclohexyl, —O-cycloheptyl, —O-cyclooctyl, —O-cyclononyl and —O-cyclodecyl, —O—CH2-cyclopropyl, —O—CH-cyclobutyl, —O—CH-cyclopentyl, —O—CH₂-cyclohexyl, —O—CH₂-cycloheptyl, —O—CH₂-cyclooctyl, —O—CH₂-cyclononyl, —O—CH₂-cyclodecyl, —O—(CH₂)₂-cyclopropyl. —O—(CH₂)₂-cyclobutyl, —O—(CH₂)₂-cyclopentyl, —O—(CH₂)₂-cyclohexyl, —O—(CH₂)₂-cycloheptyl, —O—(CH₂)₂-cyclooctyl, —O—(CH₂)₂-cyclononyl and —O—(CH₂)₂-cyclodecyl.

The term “cycloalkyl”, as used herein, unless otherwise indicated, includes a non-aromatic, saturated or partially saturated, monocyclic or fused, spiro or unfused bicyclic or tricyclic hydrocarbon referred to herein containing a total of from 3 to 10 carbon atoms, preferably 3 to 8 ring carbon atoms. Examples of cycloalkyls include, but are not limited to, C—Cs cycloalkyl groups include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl.

The term “cycloalkyl” also includes -lower alkyl-cycloalkyl, wherein lower alkyl and cycloalkyl are as defined herein. Examples of -lower alkyl-cycloalkyl groups include, but are not limited to. —CH₂-cyclopropyl. —CH₂-cyclobutyl, —CH₂-cyclopentyl, —CH₂-cyclopentadienyl, —CH₂ cyclohexyl, —CH₂-cycloheptyl and —CH₂-cyclooctyl.

The term “heterocyclic”, as used herein, unless otherwise indicated, includes an aromatic or non-aromatic cycloalkyl in which one to four of the ring carbon atoms are independently replaced with a heteroatom from the group consisting of O, S and N. Representative examples of a heterocycle include, but are not limited to, benzofuranyl, benzothiophene, indolyl, benzopyrazolyl, coumarinyl, isoquinolinyl, pyrrolyl pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H imidazolyl and tetrazolyl. Heterocycles can be substituted or unsubstituted. Heterocycles can also be bonded at any ring atom (i.e., at any carbon atom or heteroatom of the hetero cyclic ring).

The term “cyano”, as used herein, unless otherwise indicated, includes a —CN group.

The term “alcohol”, as used herein, unless otherwise indicated, includes a compound in which the hydroxyl functional group (—OH) is bound to a carbon atom. In particular, this carbon center should be saturated, having single bonds to three other atoms.

The term “solvate” is intended to mean a solvate form of a specified compound that retains the effectiveness of such compound. Examples of solvates include compounds of the invention in combination with, for example: water, isopropanol, ethanol, methanol, dimethylsulfoxide (DMSO), ethyl acetate, acetic acid, or ethanolamine.

The term “mmol”, as used herein, is intended to mean millimole.

The term “equiv”, as used herein, is intended to mean equivalent.

The term “mL, as used herein, is intended to mean milliliter.

The term “g”, as used herein, is intended to mean gram.

The term “kg”, as used herein, is intended to mean kilogram.

The term “ug”, as used herein, is intended to mean micrograms.

The term “h”, as used herein, is intended to mean hour.

The term “min”, as used herein, is intended to mean minute.

The term “M”, as used herein, is intended to mean molar.

The term “u”, as used herein, is intended to mean microliter.

The term “uM”, as used herein, is intended to mean micromolar.

The term “nM”, as used herein, is intended to mean nanomolar.

The term “N”’, as used herein, is intended to mean normal.

The term “amu”, as used herein, is intended to mean atomic mass unit.

The term “C.”, as used herein, is intended to mean degree Celsius.

The term “wt/wt”, as used herein, is intended to mean weight/weight.

The term “v/v’, as used herein, is intended to mean volume/volume.

The term “MS”, as used herein, is intended to mean mass spectroscopy.

The term “HPLC”, as used herein, is intended to mean high performance liquid chromatograph.

The term “RT”, as used herein, is intended to mean room temperature.

The term “e.g.”, as used herein, is intended to mean example.

The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate. succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis (2-hydroxy-3-naphthoate)) salts. A pharmaceutically accept able salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counterions. Hence, a pharmaceutically acceptable salt can have one or more charged atoms and/or one or more counterion.

As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine.

As used herein, the expression “pharmaceutically acceptable hydrate” refers to a compound of the invention, or a salt thereof, that further includes a stoichiometric or non-stoichiometric amount of water bound by non-covalent intermolecular forces.

### DESCRIPTION OF EMBODIMENTS

A novel flow chemical process for manufacturing compounds which absorb UV radiation and protect biological materials as well as non-biological materials from damaging UV radiation will be described hereinafter.

### Example 1: SPC-0097 Batch Formation of EK-2a™

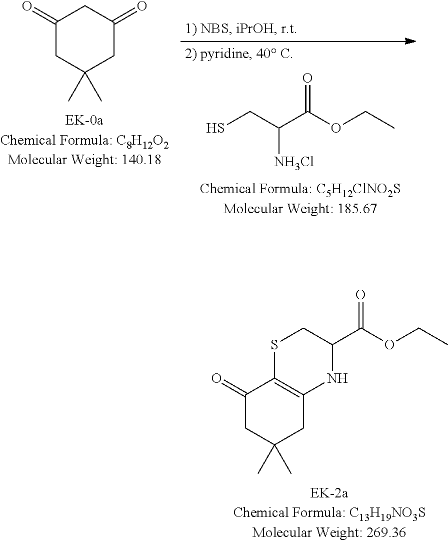

A 2 L round bottom flask was charged with 62.5 g of dimedone (EK-0a), 500 mL of 2-propanol (IPA) and a magnetic stir bar. NBS (92.5 g) was added portion wise over a 10-minute period. The mixture was heterogenous and yielded a white slurry. The mixture was stirred for 20 minutes. Pyridine (80 mL) was added to the mixture and cysteine ethyl ester HCl (100 g) was added portion wise keeping internal temperature below 30° C. The mixture turned red and homogeneous and was heated at 40° C. for 2 hours. After 2 hours at 40° C., the red solution was concentrated to about 250 mL volume. Crystals started to precipitate, the slurry was poured into 1 L of distilled water and stirred for 5 minutes before being filtered on a frit and washed with 250 mL of distilled water. The crude was recrystallized with 150 mL IPA, crystallization occurred upon cooling to room temperature, further crystallization occurs when the mixture was placed in the freezer overnight. The crystalline solid was collected on a frit and washed with 100 mL of IPA and dried in air. Yield 65.6 g (54.6%).

### Example 2: SPC-0059 Flow Formation of EK-2a

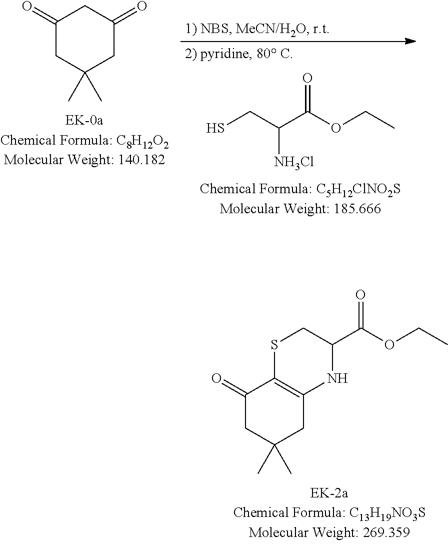

A 500 mL round bottom flask (RBF #1) was charged with 20 g of dimedone (EK-0a), 31.7 g of NBS, 387 mL acetonitrile (MeCN) and 15 mL of distilled water. The mixture is sonicated 15 minutes to afford a homogeneous solution. A 250 mL round bottom flask (RBF #2) was charged with 31.9 g of L-cysteine ethyl ester hydrochloride, 30.5 mL of pyridine, 54 mL of MeCN and 54 mL of distilled water. The two mixture were eluted through a Vapourtec continuous flow setup using a modified HPLC pump. The RBF #1 was pumped at 1.136 mL/min in a coil reactor of 10 mL (8.8 minutes) heated at ambient temperature. After the first reactor the RBF #2 was added at 0.404 mL/min to mix with the first solution and react in 4 coils of 10 mL (25.9 minutes) heated at 80° C. The reaction mixture was collected at the end of the reactor in a bottle. The mixture was concentrated by removing solvent under reduced pressure. Crystals started to precipitate, the slurry was poured into distilled water and stirred for 5 minutes before being filtered on a frit and washed with 100 mL of distilled water. The crude was recrystallized with 50 mL IPA, crystallization occurred upon cooling to room temperature, further crystallization occurs when the mixture was placed in the freezer overnight. The crystalline solid was collected on a fit and washed with 20 mL of IPA and dried in air. Yield 9 g (23%).

### Example 3: SPC-0087 Formation of EK-14-1™

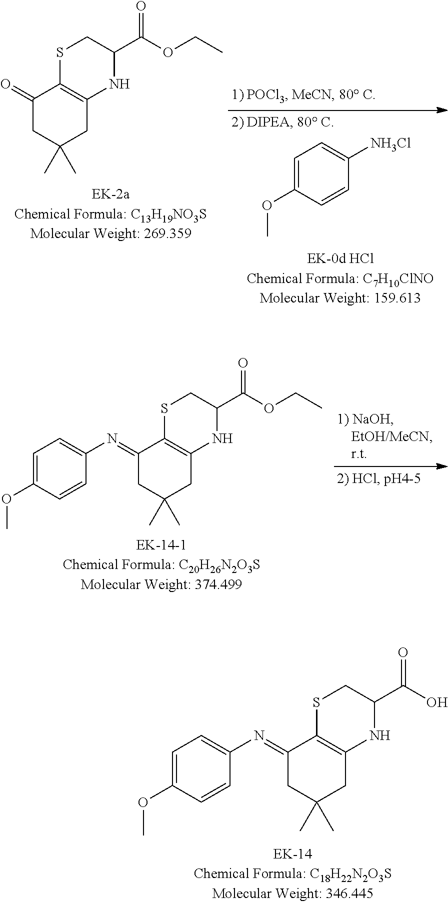

A 500 mL round bottom flask (RBF #1) was charged with 30 g of EK-2a and 346 mL of anhydrous acetonitrile (MeCN). The mixture was purged under nitrogen. 11.52 mL of POCl₃ was added to RBF #1. A second 500 mL round bottom flask (RBF #2) was charged with 17.78 g of p-anisidine HCl (EK-0dHCl), 44.6 mL of N,N-diisopropylethylamine (DIPEA) and 223 mL of MeCN. The two mixture were eluted through a Vapourtec continuous flow setup using a modified HPLC pump. The RBF #1 was pumped at 3.333 mL/min in a coil reactor of 10 mL (3 minutes) heated at 80° C. After the first reactor the RBF #2 was added at 2.22 mL/min to mix with the first solution and react in 3 coils of 10 mL (5.43 minutes) heated at 80° C. The reaction mixture was collected at the end of the reactor in a bottle. The solution was concentrated to dryness, the residue was solubilised in dichloromethane (DCM) and washed 3 times with distilled water. The organic phase was concentrated to dryness, to afford the crude EK-14-1.

### Example 4: Saponification of EK-14-1 to EK-14™

A 1 L round bottom flask was charged with the crude mixture of EK-14-1 and 200 mL of acetonitrile (MeCN). The solution was purged under nitrogen. A solution of NaOH (45 g) in ethanol (400 mL) was added and the mixture was stirred for 1 hour at ambient temperature. After 1 h, the mixture was acidified with HCl (60 mL) to reach pH 4-5 and stirred under inert atmosphere. The mixture was filtered to remove a solid (NaCl) and washed with 100 mL of MeCN. The filtrate was concentrated under reduced pressure to afford a brown oil. Azeotropic drying was performed by adding 100 mL MeCN and evaporated to dryness, to give a semi solid which started to crystallize upon cooling to RT. The product was crystallized in hot MeCN and afforded 29 g (75%) of EK-14 (in 3 crops).

### Example 5: SPC-0096 Formation of EK20-1™ and Saponification to EK-20™

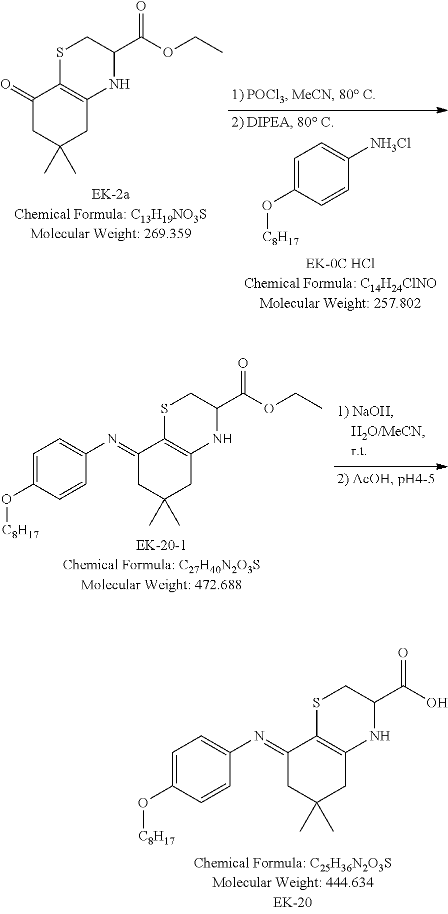

A 500 mL round bottom flask (RBF #1) was charged with 15 g of EK-2a and 173 mL of anhydrous acetonitrile (MeCN). The mixture was purged under nitrogen. 5.76 mL of POCl₃ was added to RBF #1. A second 500 mL round bottom flask (RBF #2) was charged with 14.35 g of 4-(octyloxy)anilineHCl (EK-0cHCl), 22.3 mL of N,N-diisopropylethylamine (DIPEA) and 111 mL of MeCN. The two mixture were eluted through a Vapourtec™ continuous flow setup using modify HPLC pump. The RBF #1 was pumped at 3.333 mL/min in a coil reactor of 10 mL (3 minutes) heated at 80° C. After the first reactor the RBF #2 was added at 2.22 mL/min to mix with the first solution and react in 3 coils of 10 mL (5.43 minutes) heated at 80° C. The reaction mixture was collected at the end of the reactor in a bottle. A 1 L round bottom flask was charged with the mixture of EK-20-1 and purged under nitrogen. A solution of NaOH (24.5 g) in water (240 mL) was added and the mixture was stirred for 1 hour at ambient temperature. After 1 h, the mixture was acidified with acetic acid (35 mL) to reach pH 4-5 and stirred under inert atmosphere. The mixture was poured into distilled water (800 mL) and started to precipitate, the mixture was cooled in fridge for 3 h. The product was isolated by filtration and washed with 150 mL of distilled water. Dried overnight and recrystallized in 100 mL THF afforded 11 g (42%) of EK-20 in a bright yellow solid.

### Example 6: Properties of Synthesized EK™ Compounds

The compounds derived from the processes referenced above were found to provide protection against UVA, UVB, UV-visible, infra-red and blue rays. In addition, the compounds showed interesting properties as antioxidants, as moisturizers and as topical anti-inflammatory agents. Advantageously, the compounds are nontoxic.

The compounds derived from the processes referenced above will find use in the following fields: cosmetics, beauty and personal care, and textiles (aramids and special fibers), plastic and polyester and polyethylene films. The compounds act by providing a protective coating against harmful UV rays and other forms of radiation.

### Example 7: SPC-0191 Telescoping of EK-20 in Batch

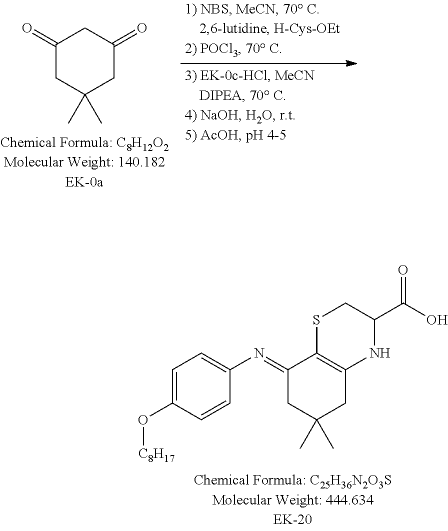

A 1 L round bottom flask was charged with 5 g of dimedone (EK-0a), 7.62 g of NBS, 23 mL of acetonitrile (MeCN) and magnetic stir bar under inert atmosphere (N₂). At room temperature (r.t.), 2,6-lutidine (12 mL) was added followed by L-cysteine ethyl ester hydrochloride (6.62 g). The reaction mixture was stirred at 70° C. for 1 hour to afford high conversion to intermediary EK-2a. POCl₃ (5 mL) was added dropwise and the reactions was stirred 10 minutes at 70° C., affording high conversion to intermediary EK-3a. A solution containing EK-0c-HCl (9.2 g), N,N-diisopropylethylamine (14 mL) and MeCN (7 mL) was added portion-wise to the reaction mixture and stirred 10 minutes at 70° C. to afford high conversion of EK-20-1 (ester). A saponification was carried out using a 20% solutions of NaOH (20 eq.) in water, the reaction mixtures was stirred at r.t. for 1.25 hours to afford total conversion of EK-20-1 in EK-20. The reactions mixture was acidified with acetic acid (20 mL) to a pH of 4-5 and water (500 mL) was added to precipitate the product out. The product was isolated by filtration, washed, dried overnight and recrystallized to afford 8 g (51%) of EK-20 in a bright yellow solid.

### Example 8: SLB39 Batch Formation of EK17-1™

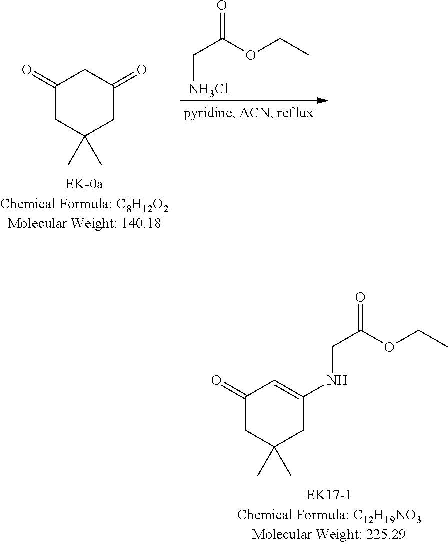

Dimedone (35.0 g), glycine ethyl ester hydrochloride (38.5 g) and pyridine (25 ml) were combined in CH₃CN (400 ml). The suspension was heated to reflux and stirred overnight. The solvent was removed under vacuum and the resulting yellow oil was diluted in CH₂Cl₂ (250 ml), extracted with one portion of water (250 ml) and brine (250 ml), and dried with MgSO₄. The solvent was evaporated and the solid was crystallized in hot ACN to yield EK17-1 (35.0 g, 3 crops, 62%) as colorless needles.

### Example 9: SLB38 Batch Formation of EK17-3™

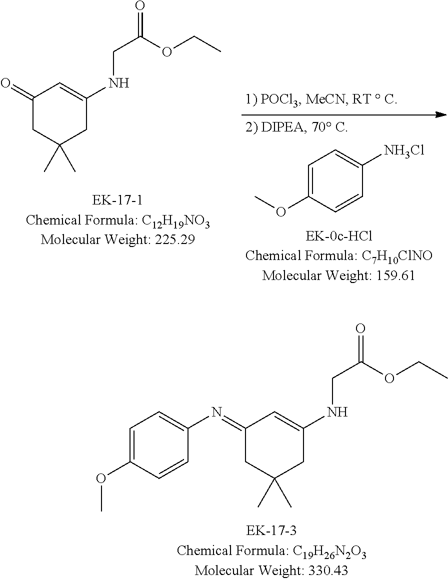

Under inert atmosphere, POCl₃ (4.5 ml) was added to a solution of EK17-1 (9.48 g) in dry ACN (50 ml). The solution was stirred at room temperature for 1 h30, heated to 70° C. and stirred for an additional 30 minutes. A solution of anisidine HCl (7.50 g) and DiPEA (17 ml) in dry ACN (50 ml) was transferred to the reaction mixture and the solution was stirred at 70° C. for an hour. Additional portions of anisidine HCl (765 mg) and DiPEA (2.0 ml) were added to ensure complete conversion of the starting material. ACN was removed under vacuum and the resulting orange oil was diluted in CH₂Cl₂ (50 ml), extracted with water and brine, and dried with MgSO₄. The solvent was evaporated and addition of ethyl acetate (50 ml) to the resulting orange oil lead the formation of a solid. The product was isolated by filtration and the filtrate was evaporated. The cycle of aqueous extractions followed by precipitation in ethyl acetate and separation by filtration were repeated twice. The combined solids were recrystallized in hot ACN to yield EK17-3 (8.65 g, 62%) as a pale yellow solid.

### Example 10: SLB43 Batch Formation of EK17™

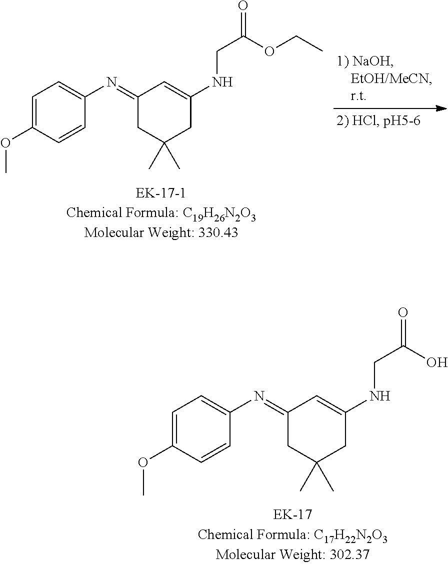 [SMILES: CCOC(=O)CNC1=C/C(=N/c2ccc(OC)cc2)CC(C)(C)C1 ; COc1ccc(/N=C2/C=C(NCC(=O)O)CC(C)(C)C2)cc1]

EK17-03 (10.4 g) was suspended in ACN (60 ml) and a solution of NaOH (60 ml, 0.125 g/ml) in ethanol was added. The reaction mixture was stirred at room temperature for an hour. The suspension was acidified with concentrated HCl to pH 5.3 and filtered. The solvent was partially evaporated under vacuum before adding ACN (35 ml) which induced crystallization. The suspension was filtered, and the residue was rinsed with ACN to isolate EK17 (7.6 g, 2 crops, 80%) as a pale yellow solid.

Novel formulations that contain compounds which absorb UV radiation and protect biological materials as well as non-biological materials from damaging UV radiation will be described hereinafter.

Basic Neutral Cream: A basic neutral cream was developed in order to test how the above-specified EK™ actives behave—alone without any combination with commercial SPF actives—in cosmetic formula. Particularly to evaluate their stability, their SPF and their absorbance.

TABLE 1
Base of neutral cream

| | | Percentage | | |
|---|---|---|---|---|
| Functions | INCI | Range | | |
| Basic | WATER | 70-80 | | |
| Ingredients | GLYCERIN | 0-5 | | |
| | SODIUM POLYACRYLATE | 0-1 | | |
| | XANTHAN GUM | 0-1 | | |
| | CAPRIC/CAPRYLIC TRYGLYCERIDES | 5-10 | | |
| | HELIANTHUS ANNUUS (SUNFLOWER | 0-5 | | |
| | SEED) OIL | | | |
| | CETEARYL ALCOHOL | 0-5 | | |
| | GLYCERYL STEARATE SE | 0-5 | | |
| | PHENOXYETHANOL, SORBIC ACID, | 0-1 | | |
| | CAPRYLYL GLYCOL | | | |
| Actives | EK-14 | 0.5 | 0 | 0 |
| | EK-17 | 0 | 0.5 | 0 |
| | EK-20 | 0 | 0 | 0.5 |

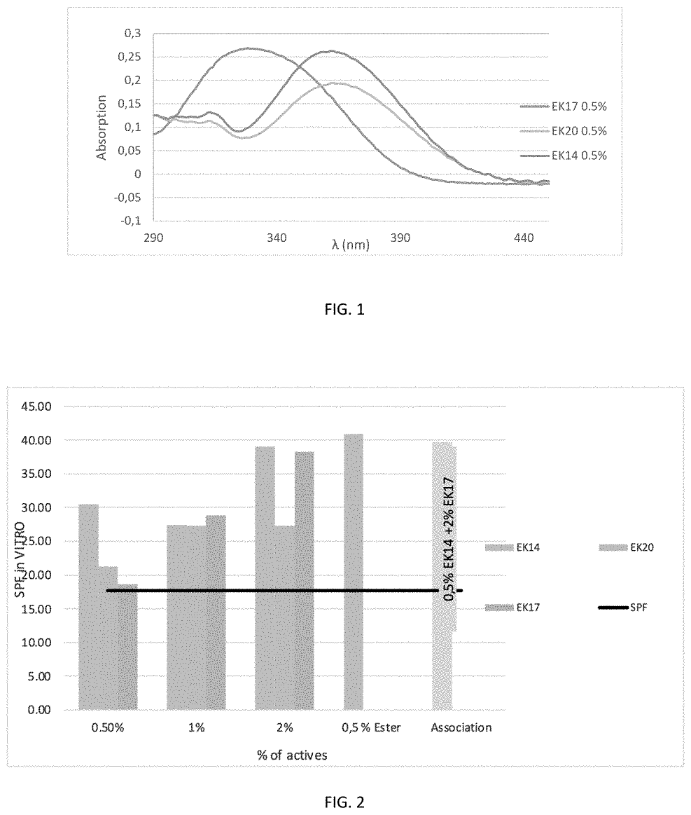

Despite their normal SPF activity, EK actives show great stability in formulations at a good range of temperature (5-45 Celsius). They also show excellent UV absorption as shown in FIG. 1. This Figure also shows that EK-17 absorbs mainly in UVB while EK-14 and EK-20 absorb mainly in UVA.

EK actives were tried in combination of existing solar filters on several concentration in order to obtain SPF, UVA, between SP15 and SP60+. Table 2 shows examples and results of a formulation with an SPF 30. EK-actives show clearly an SPF boosting effect from 5% till 125% depending on concentration and combination. EK-14 is the most potent EK-active since it shows a gain of 72% of SPF at 0.5% concentration.

TABLE 1
Sunscreen base cream with common solar filters and EK-actives

| Fonctions | INCI | Percentage range | | | | | | | | | | | |
|---|---|---|---|---|---|---|---|---|---|---|---|---|---|
| Basic | WATER | 60-70 | | | | | | | | | | | |
| ingredients | GLYCERIN | 0-5 | | | | | | | | | | | |
| | SODIUM | 0-1 | | | | | | | | | | | |
| | POLYACRYLATE | | | | | | | | | | | | |
| | XANTHAN GUM | 0-1 | | | | | | | | | | | |
| | CAPRIC/CAPRYLIC | 5-10 | | | | | | | | | | | |
| | TRYGLYCERIDES | | | | | | | | | | | | |
| | DICAPRYLYL | 0-5 | | | | | | | | | | | |
| | CARBONATE | | | | | | | | | | | | |
| | CETEARYL ALCOHOL | 0-5 | | | | | | | | | | | |
| | GLYCERYL STEARATE, | 0-5 | | | | | | | | | | | |
| | PEG-100 STEARATE | | | | | | | | | | | | |
| | PHENOXYETHANOL, | 0-1 | | | | | | | | | | | |
| | SORBIC ACID, | | | | | | | | | | | | |
| | CAPRYLYL GLYCOL | | | | | | | | | | | | |
| Solar | DIETHYLAMINO | 5-10 | | | | | | | | | | | |
| filte | HYDROXYBENZOYL | | | | | | | | | | | | |
| | HEXYL BENZOATE | | | | | | | | | | | | |
| | ETHYLHEXYL | 0-5 | | | | | | | | | | | |
| | TRIAZONE | | | | | | | | | | | | |
| | ETHYLHEXYL | 5-10 | | | | | | | | | | | |
| | METHOXYCINNAMATE | | | | | | | | | | | | |
| Actives | EK 14 | 0.5 | | | | | | | | | | | |
| | | | 1 | | | | | | | | | | |
| | | | | 2 | | | | | | | | | |
| | | | | | | | | | | | | 0.5 | |
| | EK14-1 | | | | 0.5 | | | | | | | | |
| | EK17 | | | | | 0.5 | | | | | | | |
| | | | | | | | 1 | | | | | | |
| | | | | | | | | 2 | | | | | |
| | | | | | | | | | | | | 2 | 2 |
| | EK20 | | | | | | | | 0.5 | | | | |
| | | | | | | | | | | 1 | | | |
| | | | | | | | | | | | 2 | | |
| | | | | | | | | | | | | | 0.5 |

| | | | | | | | | | | | | |
|---|---|---|---|---|---|---|---|---|---|---|---|---|
| Evolution (in %) FPS in vitro | +72% | +55% | +120% | +131% | +5% | +63% | +116% | +20% | +54% | +54% | +124% | +115% |
| versus control without active | | | | | | | | | | | | |

| Basic | WATER | 60-70 | | | | | | | | | | | |
|---|---|---|---|---|---|---|---|---|---|---|---|---|---|
| Ingredients | GLYCERIN | 0-5 | | | | | | | | | | | |
| | SODIUM | 0-1 | | | | | | | | | | | |
| | POLYACRYLATE | | | | | | | | | | | | |
| | XANTHAN GUM | 0-1 | | | | | | | | | | | |
| | CAPRIC/CAPRYLIC | 5-10 | | | | | | | | | | | |
| | TRYGLYCERIDES | | | | | | | | | | | | |
| | DICAPRYLYL | 0-5 | | | | | | | | | | | |
| | CARBONATE | | | | | | | | | | | | |
| | CETEARYL ALCOHOL | 0-5 | | | | | | | | | | | |
| | GLYCERYL STEARATE, | 0-5 | | | | | | | | | | | |
| | PEG-100 STERATE | | | | | | | | | | | | |
| | PHENOXYETHANOL, | 0-1 | | | | | | | | | | | |
| | SORBIC ACID, | | | | | | | | | | | | |
| | CAPRYLYL GLYCOL | | | | | | | | | | | | |
| | TOCOPHEROL | 0-1 | | | | | | | | | | | |
| Solar | DIETHYL AMINO | 5-10 | | | | | | | | | | | |
| Filters | HYDROXYBENZOIL | | | | | | | | | | | | |
| | HEXYL BENZOATE | | | | | | | | | | | | |
| | ETHYHEXYL | 0-5 | | | | | | | | | | | |
| | TRIAZONE | | | | | | | | | | | | |
| | ETHYHEXYL | 5-10 | | | | | | | | | | | |
| | METHOXYCINNAMATE | | | | | | | | | | | | |
| Actives | EK-14 | 0.5 | | | | | | | | | | | |
| | | | 1 | | | | | | | | | | |
| | | | | 2 | | | | | | | | | |
| | | | | | | | | | | | | 0.5 | |
| | EK-14-1 | | | | 0.5 | | | | | | | | |
| | EK-17 | | | | | 0.5 | | | | | | | |
| | | | | | | | 1 | | | | | | |
| | | | | | | | | 2 | | | | | |
| | | | | | | | | | | | | 2 | 2 |
| | EK-20 | | | | | | | | 0.5 | | | | |
| | | | | | | | | | | 1 | | | |
| | | | | | | | | | | | 2 | | |
| | | | | | | | | | | | | | 0.5 |

| | | | | | | | | | | | |
|---|---|---|---|---|---|---|---|---|---|---|---|
| Evolution (%) FPS in vitro | +72 | +55 | +120 | +131 | +5 | +63 | +116 | +20 | +54 | +54 | +124 |
| vs control without active | | | | | | | | | | | |

FIG. 2 illustrates the SPF advantage of EK-actives on different concentration. It is noteworthy that EK-active esters are more potent than EK-active acids. Also, a combination of 2 or more EK-actives show very interesting SPF boosting effect considering that such combination will assure wide UV range protection.

EK actives were tried in combination of existing mineral (zinc Oxide and Titanium dioxide) with solar filters on several concentration in order to obtain SPF, UVA, between SP15 and SP60+. Table 3 shows examples and results of a formulation with an SPF 30. EK-actives show promising preliminary results at different concentration.

TABLE 3
Sunscreen base cream with common mineral solar filters

| | | Percentage |
|---|---|---|
| Fonctions | INCI | range |
| Basic | WATER | 50-60 |
| Ingredients | CAPRIC/CAPRYLICTRYGLYCERIDES | 5-10 |
| | UNDECANE (AND) TRIDECANE | 5-10 |
| | POLYGLYCERYL-6 | 0-5 |
| | POLYHYDROXYSTEARATE (AND) | |
| | POLYGLYCERYL-6 POLYRICINOLEATE | |
| | TOCOPHEROL | 0-1 |
| | PHENOXYETHANOL, SORBIC ACID, | 0-1 |
| | CAPRYLYL GLYCOL | |
| | GLYCERIN | 0-5 |
| | MAGNESIUM SULFATE | 0-1 |
| Solar | ZINC OXIDE | 10-15 |
| filters | TITANIUM DIOXYDE | 0-5 |

EK actives were tried in a special formulation that combines existing solar filters and additives on several concentrations. This formulation was applied on special fabrics such as aramids and Kevlar. Table 4 shows examples of such formulation with different concentrations.

TABLE 4
Example of Formulation applied on
surface of special fabric such aramids

| | Formu- | Formu- | Formu- | Formu- |
|---|---|---|---|---|
| Product | lation | lation | lation | lation |
| MeOH | 10% | 10% | 4% | 20%, 25% and 30% |
| Propylene | 10% | 10% | 2.6% | |
| Glycol | | | | |
| Eversorb | 2% | 2% | 0.5% | |
| Dowanol | 3% | 3% | 0.4% | |
| Water | 55% | 55% | 87.6% | 20%, 25%, 30%, 35%, 40% |
| Butyl Carbitol | 0% | 0% | 0.8% | |
| Pekoflam | 15% | 15% | 3.5% | 20%, 25%, 30% |
| suncure | | | | 10%, 25% |
| Parsol | | | | 5% and 10% |
| Emulgin | | | | 2.5%, 5% and 10% |
| EK-14 | 5% | 0 | 0 | 10%, 15% and 20% |
| EK-17 | 0 | 5% | 0.64% | 10%, 15% and 20% |
| EK-19 | | | | 10%, 15% and 20% |

TABLE 5
Example on EK-actives activity on special textile and fabrics

| | | | Tests for | | | |
|---|---|---|---|---|---|---|
| | | | Colourfastness | | | |
| | | | (100 Hrs) | Breaking Strength of Fabrics- | | |
| | Pick-Up | | CAN/CGSB- | Strip Method-Constant- | | |
| | Level | | 4.2, 18.3 | time-to-break Principle | | |
| | (% weight, | | Grade 1 to 5 | (method CAN/CGSB-4.2, 9.1-M90) | | |
| Sample | w/w) | Washing | (5 is excellent) | Before | After | Breaking |
| EK14-006 | 32.7 | | 2 | 1239 | 834 | 33% |
| EK17-007 | 39.2 | | 03-févr | 1119 | 766 | 41% |
| EK14-006 | 39.4 | 5 | 3 | 1332 | 604 | 55% |
| EK17-006 | 39.2 | 5 | 03-févr | 1316 | 494 | 63% |
| EK-14 + Dyed | | 5 | | 1119 | 651 | 41% |
| Fabric | | | | | | |

TABLE 6
Example of activity of EK-17 on special aramid fabric

| | | Tear resistance | | | | | | |
|---|---|---|---|---|---|---|---|---|
| | | ASTM D5587 | | | | | | |
| | | | | | | | | after UV, |
| | | | | | after UV, | | after 5 | 100 hrs |
| | | | | | 100 hrs | | washes | (CAN/CGSB- |
| | | | | | (CAN/CGSB- | | AATCC 135 | 4.2, 18.3) |
| | | | | after 5 | 4.2, 18.3) | after UV, | method 1 ,v,Ai | and 5 washes |
| | | | after UV, | washes | and 5 washes | 100 hrs | AATCC 135 | AATCC 135 |
| | | | 100 hrs | AATCC 135 | AATCC 135 | (CAN/CGSB- | method | method |
| | | | (CAN/CGSB- | method | method | 4.2, 18.3) | 1, V, Ai | 1, V, Ai |
| Sample | | initial | 4.2, 18.3) | 1, V, Ai | 1, V, Ai | % retention | % retention | % retention |
| ID | direction | (N) | (N) | (N) | (N) | to initial | to initial | to initial |
| TC-EK17-12-oct | length | 259 | 189 | 218 | 225 | 73 | 84 | 87 |
| TC (control) | length | 944 | 458 | 279 | 296 | 49 | 30 | 31 |
| TC-EK17-12-oct | width | 566 | 297 | 343 | 453 | 52 | 61 | 80 |
| TC (control) | width | 815 | 303 | 351 | 347 | 37 | 43 | 43 |

As shown in tables 5 and 6, EK actives protect fabrics from UV radiation in a wide concentration range between 0.35%-40%. This protection lasts after UV aging and after several washing cycles. EK actives were added using several application techniques including film application, dyed fabric application and thread application techniques.

UV Protection Effect

EK actives were tried in a special formulation that combines existing solar filters and additives on several concentration. This formulation was applied on special coating films as shown in table 7.

Table 7: Example of Formulation of special coating applied on plastic and metal surface.

TABLE 8
Example of activity of EK-17 on special coating film

| Product | Formulation | | | | Formulation | | | | formulation | |
|---|---|---|---|---|---|---|---|---|---|---|
| Impranil 1554 | 100 | | | | 100 | | | | 100 | |
| BYK 333 | 0.4 | | | | 0.4 | | | | 0.4 | |
| Borchigel L75 N | 1.05 | | | | 10.5 | | | | 0.9 | |
| (25% in DI water | | | | | | | | | | |
| Borchigel ALA | 0.68 | | | | 0.68 | | | | 1.15 | |
| EK-17 | 1.05 | | | | 0 | | | | 0 | |
| EK-21 | 0 | | | | 0.55 | | | | 0 | |
| EK-14 | 0.5 | | | | 1 | | | | 2 | |
| | T0 | | T1 (0.5% EK-17) | | T2 (1% Ek-17) | | T3 (1.5% Ek-17) | | T4 | |
| | Before | After | Before | After | Before | After | Before | After | Before | After |
| 100% Modulus | 2.86 | — | 2.99 | 2.47 | 3.08 | 2.54 | 3.4 | 2.78 | 2.72 | 2.42 |
| (MPa) | | | | | | | | | | |
| Tensile Strength | 24.57 | — | 18.61 | 20 | 23.15 | 26.67 | 26.09 | 28.05 | 20.46 | 28.14 |
| (MPa) | | | | | | | | | | |
| Elongation at | 909 | — | 767 | 795 | 853 | 945 | 923 | 932 | 1008 | 932 |
| Break (%) | | | | | | | | | | |

As shown in table 8, EK actives protect special coating from UV radiation in a wide concentration range between 0.5%-1.5%. This protection lasts after UV aging and shows enhancement of material performance.

While illustrative and presently preferred embodiments of the invention have been described in detail hereinabove, it is to be understood that the inventive concepts may be otherwise variously embodied and employed and that the appended claims are intended to be construed to include such variations except insofar as limited by the prior art.

### REFERENCES

ⁱ. Cardozo et al. 2007. Metabolites from algae with economical impact. Comparative Biochemistry and Physiology Part C: Toxicology & Pharmacology, Volume 146, Issues 1-2: 60-78. ⁱⁱBandaranayake W. M. 1998. Mycosporines: are they nature's sunscreens? Natural Product Reports. 15(2): 159 72.

1. A method of producing an ultraviolet radiation absorbing compound in flow conditions, the method comprising the steps of:
a. providing a source of dimedone;
b. providing a source of IPA;
c. forming a reaction mixture by adding portion wise NBS to form a white slurry;
d. stirring the white slurry;
e. forming a reaction mixture by adding pyridine and adding portion wise cysteine ethyl ester HCl while maintaining an internal temperature below 30° C.;
f. heating the mixture at 40° C. until the reaction mixture is red; and
g. crystalizing the reaction mixture to form the ultraviolet radiation absorbing compound.

2. The method of claim 1 further comprising recrystalizing with IPA.

3. The method of claim 1 further comprising cooling the mixture.

4. The method of claim 1 further comprising the steps of:
a. adding MeCN to form a first mixture;
b. purging the first mixture under nitrogen;
c. adding POCl₃ to the first mixture;
d. providing a source of p-anisidine HCl, N,N-diisopropylethylamine and MeCN into a second mixture;
e. eluting the first and second mixtures into a final mixture;
f. forming crystals by concentrating the final mixture.

5. The method of claim 4 wherein the first and second mixtures are heated to 80° C. during the elution.

6. The method of claim 4 further comprising concentrating the final mixture to dryness.

7. The method of claim 4 further comprising the steps of:
a. adding MeCN to form a third mixture;
b. purging the third mixture under nitrogen;
c. adding a solution of NaOH and ethanol;
d. stirring the third mixture;
e. acidifying the third mixture with HCl to reach a pH level between 4 and 5;
f. stirring under inert atmosphere;
g. filtering the third mixture to remove NaCl;
h. washing the third mixture with MeCN;
i. concentrating under reduced pres and
j. evaporating to dryness.

8. The method of claim 1 further comprising the steps of:
a. adding MeCN to form a first mixture;
b. purging the first mixture under nitrogen;
c. adding POCl₃ to the first mixture;
d. providing a source of 4-(octyloxy)anilineHCl (EK-0cHCl), N,N-diisopropylethylamine and MeCN into a second mixture;
e. eluting the first and second mixtures into a third mixture;
f. purging the final mixture under nitrogen;
g. adding a solution of NaOH and water to the third mixture to form a final mixture;
h. stirring the final mixture;
i. acidifying the final mixture with acetic acid to a pH between 4 and 5;
j. cooling the final mixture; and
k. filtering the final mixture.

9. The method of claim 8 wherein the first and second mixtures are heated to 80° C. during the elution.

10. A method of producing an ultraviolet radiation absorbing compound in flow conditions, the method comprising the steps of:
a. providing a source of dimedone, NBS, MeCN and distilled water into a first mixture;
b. forming a homogeneous solution of the first mixture;
c. providing a source of L-cysteine ethyl ester hydrochloride, pyridine, MeCN and distilled water into a second mixture;
d. eluting the first and second mixtures into a final mixture;
e. removing the solvent under reduced pressure; and
f. forming crystals by concentrating the final mixture.

11. The method of claim 10 further comprising forming a homogenous solution of the first mixture by sonication.

12. The method of claim 10 wherein the second mixture is heated to 80° C. during the elution.

13. The method of claim 10 further comprising stirring the final mixture in distilled water.

14. The method of claim 10 further comprising recrystalizing with IPA.

15. The method of claim 10 further comprising cooling the mixture.

16. The method of claim 10 further comprising the steps of:
a. adding MeCN to form a first mixture;
b. purging the first mixture under nitrogen;
c. adding POCl₃ to the first mixture;
d. providing a source of p-anisidine HCl, N,N-diisopropylethylamine and MeCN into a second mixture;
e. eluting the first and second mixtures into a final mixture;
f. forming crystals by concentrating the final mixture.

17. The method of claim 16 wherein the first and second mixtures are heated to 80° C. during the elution.

18. The method of claim 16 further comprising concentrating the final mixture to dryness.

19. The method of claim 16 further comprising the steps of:
a. adding MeCN to form a third mixture;
b. purging the third mixture under nitrogen;
c. adding a solution of NaOH and ethanol;
d. stirring the third mixture;
e. acidifying the third mixture with HCl to reach a pH level between 4 and 5;
f. stirring under inert atmosphere;
g. filtering the third mixture to remove NaCl;
h. washing the third mixture with MeCN;
i. concentrating under reduced pres and
j. evaporating to dryness.

20. The method of claim 10 further comprising the steps of:
a. adding MeCN to form a first mixture;
b. purging the first mixture under nitrogen;
c. adding POCl₃ to the first mixture;
d. providing a source of 4-(octyloxy)anilineHCl (EK-0cHCl), N,N-diisopropylethylamine and MeCN into a second mixture;
e. eluting the first and second mixtures into a third mixture;
f. purging the final mixture under nitrogen;
g. adding a solution of NaOH and water to the third mixture to form a final mixture;
h. stirring the final mixture;
i. acidifying the final mixture with acetic acid to a pH between 4 and 5;
j. cooling the final mixture; and
k. filtering the final mixture.

21. The method of claim 20 wherein the first and second mixtures are heated to 80° C. during the elution.

22. A method of producing an ultraviolet radiation absorbing compound in flow conditions, the method comprising the steps of:
a. providing a source of dimedone, N-Bromosuccinimidel and MeCN into a first mixture under inert atmosphere;
b. adding 2,6-lutidine and L-cysteine ethyl ester hydrochloride;
c. stirring the first mixture;
d. adding dropwise phosphoryl chloride;
e. adding dropwise a solution comprising EK-0c-HCl, N,N-diisopropylethylamine and MeCN;
f. stirring the first mixture;
g. obtaining saponification using a 20% solution of NaOH in water;
h. acidifying the first mixture with acetic acid to a pH between 4 and 5; and
i. filtering the first mixture.

23. The method of claim 22 wherein the inert atmosphere comprises dinitrogen.

24. The method of claim 22 wherein the first mixture is stirred at 70° C.

25. A method of producing an ultraviolet radiation absorbing compound in flow conditions, the method comprising the steps of:
a. combining dimedone, glycine ethyl ester hydrochloride and pyridine in acetonitrile to form a suspension;
b. heating the suspension;
c. stirring the suspension;
d. removing the acetonitrile under vacuum to form a viscous liquid;
e. diluting the viscous liquid in CH₂Cl₂;
f. extracting with water and brine;
g. drying with MgSO₄;
h. evaporating the remaining acetonitrile;
i. crystalizing the ultraviolet radiation absorbing compound in acetonitrile.

26. The method of claim 22 further comprising the steps of:
a. adding POCl₃ under inert atmosphere in dry ACN to form a first solution;
b. stirring the first solution;
c. heating the first solution to 70° C.;
d. adding a second solution comprising anisidine HCl and DiPEA in dry CAN to the first solution to form a third solution;
e. stirring the third solution at 70° C.;
f. adding anisidine HCl and DiPEA until a complete conversion of the starting material is achieved;
g. removing the ACN under vacuum to form a viscous liquid;
h. diluting in CH₂Cl₂, extracted with water and brine, and dried with MgSO₄;
i. evaporating the solvent;
j. adding ethyl acetate;
k. isolating the ultraviolet radiation absorbing compound by filtration;

27. The method of claim 23 further comprising the steps of:
a. suspending the ultraviolet radiation absorbing compound in ACN to form a suspension;
b. adding a solution of NaOH in ethanol;
c. stirring the mixture;
d. acidifying the suspension with concentrated HCl to pH 5.3;
e. filtering the suspension;
f. partially evaporating the solvent under vacuum;
g. inducing crystallization by adding CAN;
h. filtering the suspension; and
i. rinsing with ACN.

28. Use of the ultraviolet radiation absorbing compound according to any one of claims 1 to 27 for manufacturing a cream.

29. The use of the ultraviolet radiation absorbing compound according to claim 28, wherein the cream is applied to a biological material.

30. The use of the ultraviolet radiation absorbing compound according to claim 29, wherein the cream is applied to skin.

31. The use of the ultraviolet radiation absorbing compound according to claim 28, wherein the cream is a sunscreen.

32. The use of the ultraviolet radiation absorbing compound according to claim 28, wherein the cream is a moisturizing cream.

33. The use of the ultraviolet radiation absorbing compound according to claim 28, wherein the cream is an anti-aging cream.

34. The use of the ultraviolet radiation absorbing compound according to claim 28, wherein the cream is applied to a non-biological material.

35. The use of the ultraviolet radiation absorbing compound according to claim 34, wherein the cream is applied to a textile or fabric.

36. The use of the ultraviolet radiation absorbing compound according to claim 35, wherein the textile comprises aramid.

37. The use of the ultraviolet radiation absorbing compound according to claim 34, wherein the cream is applied using an application technique selected from: film application, dyed fabric application and thread application.
